(19) United States
(12) Patent Application Publication
(10) Pub. No.: US 2017/0343549 A9
(43) Pub. Date: Nov. 30, 2017

(54) METHODS FOR DETECTING AND MONITORING COLORECTAL CANCER

(71) Applicant: REGENTS OF THE UNIVERSITY OF MINNESOTA, MINNEAPOLIS, MN (US)

(72) Inventors: Zigang Dong, Austin, MN (US); Ann M. Bode, Austin, MN (US)

(21) Appl. No.: 15/371,483

(22) Filed: Dec. 7, 2016

Related U.S. Application Data
(60) Provisional application No. 62/264,648, filed on Dec. 8, 2015.

(51) Int. Cl. G01N 33/574

(52) U.S. Cl. CPC G01N 33/57419; G01N 2800/50

(57) ABSTRACT

A method for detecting and/or measuring circulating TXA₂ in a subject having or at risk of having colorectal cancer. Generally, the method includes obtaining a biological sample from the subject, measuring circulating TXA₂ in the biological sample, and identifying the subject as having colorectal cancer if the circulating TXA₂ in the biological sample is greater than a predetermined level. The method may be used as a diagnostic test and/or may be performed repeatedly to monitor the status of colorectal cancer in a subject over time.

### CROSS-REFERENCE TO RELATED APPLICATION

This application claims priority to U.S. Provisional Patent Application No. 62/264,648, filed Dec. 8, 2015, which is incorporated herein by reference.

### GOVERNMENT FUNDING

This invention was made with government support under CA166011, CA172457, and R37CA081064, each awarded by the National Institutes of Health. The government has certain rights in the invention.

### SUMMARY

This disclosure describes, in one aspect, a method for detecting and/or measuring circulating TXA₂ in a biological sample. Generally, the method includes obtaining a biological sample from a subject having or at risk of having colorectal cancer and then measuring circulating TXA₂ in the biological sample.

In some embodiments, measuring circulating TXA₂ in the biological sample involves determining the amount of TXA₂ in a biological sample that includes a blood product such as plasma.

In some embodiments, measuring circulating TXA₂ in the biological sample involves determining the amount of a TXA₂ metabolite in a biological sample non-blood product biological sample such as urine. In some of these embodiments, the TXA₂ metabolite includes 11-dehydro TXB₂.

In some embodiments, the method determining whether circulating TXA₂ is at least 1000 pg/mL.

In some embodiments, the method further involves administering therapy to the subject effective for treating colorectal cancer. In some of these embodiments, the therapy is effective to decrease circulating TXA₂.

In another aspect, this disclosure describes a method of monitoring changes in circulating TXA₂ over time. Generally, the method involves obtaining a present biological sample from a subject having colorectal cancer, measuring present circulating TXA₂ in the biological sample, obtaining a previous circulating TXA₂ value obtained from a previous biological sample obtained from the subject, and detecting a change in circulating TXA₂ between the previous biological sample and the present biological sample.

In some embodiments, the subject has undergone therapeutic treatment for colorectal cancer between obtaining the previous circulating TXA₂ value and obtaining the present biological sample.

In some embodiments, the method further involves administering therapy to the subject effective for treating colorectal cancer. In some of these embodiments, the therapy is effective to decrease circulating TXA₂.

In some embodiments, measuring circulating TXA₂ in the present biological sample involves determining the amount of TXA₂ in a blood product such as plasma. In some embodiments, measuring circulating TXA₂ in the present biological sample involves determining the amount of a TXA₂ metabolite in a non-blood product biological sample such as urine. In some of these embodiments, the TXA₂ metabolite includes 11-dehydro TXB₂.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Circulating prostaglandin (PG) biosynthesis in colorectal cancer progression. (A) Circulating PG levels in healthy subjects or familial adenomatous polyposis (FAP) patients. 1=healthy subjects (n=16); 2=FAP patients with colonic adenomas (n=24); 3=FAP patients with colonic adenocarcinomas (n=18). Serum samples were collected for measurement of circulating prostaglandin levels using an enzyme immunoassay kit (Cayman Chemical Co., Ann Arbor, Mich.). Data are presented as means ±S.D. The asterisks indicate a significant (**,p<0.01; ***, p<0.001) difference compared to the group of healthy subjects. (B) Profiles of circulating PG biosynthesis in healthy subjects (1) or sporadic colorectal cancer (2) patients (n=20). Data are presented as means ±S.D. The asterisks indicate a significant (**, p<0.01; ***, p<0.001) difference compared to the group of healthy subjects.

FIG. 2. Prognostic value of circulating TXA₂ levels in colorectal cancer. To confirm the prognostic value of circulating TXA₂ levels in colorectal cancer, a test study was conducted in healthy subjects (n=16), FAP patients (n=24), and colorectal cancer patients with (n=18) or without FAP history (n=20). Based on a value of 1000 pg/mL, which was selected as a practical cutoff point, 95% of colorectal cancer patients and 88% of FAP patients were successfully identified.

FIG. 3. Pathophysiological role of the TXA₂ pathway in colorectal cancer. (A) Immunohistochemical staining of TBXA2R, TBXAS1, or mPGES-1 in biopsy samples, which included normal colonic mucosa, polyps, adenomas, and adenocarcinomas. For antibody-negative controls, the primary antibodies were substituted with normal rabbit serum. Original magnification: 200×. (B) The TXA₂ pathway is required for tumorigenic properties in human colorectal cancer cells. Knockdown of TBXA2R or TBXAS1 in HT29 or HCT116 colon cancer cells was analyzed by Western blot (upper panels). Mock and knockdown HT29 and HCT116 colon cancer cells were then subjected to anchorage-independent growth assays (lower bar graphs) as described in Example 1. The asterisks (***) indicate a significant (p<0.001) decrease in colony formation by knockdown HT29 or HCT116 colon cancer cells.

FIG. 4. Aspirin reduces colorectal cancer risk in FAP patients by targeting the TXA₂ pathway. (A) Effects of regular aspirin use on circulating PG biosynthesis in FAP patients. 1=healthy subjects (n=16); 2=FAP patients, aspirin nonusers (n=24); 3=FAP patients, aspirin users (n=14). FAP patients who reported taking two or more standard (325 mg) aspirin tablets per week were classified as regular aspirin users and those taking less aspirin were defined as aspirin nonusers. Data are presented as means ±S.D. The asterisks (***) indicate a significant (p<0.001) decrease in circulating TXA₂ levels associated with aspirin intake. (B) Effects of regular aspirin use on the expression patterns of TBXA2R, TBXAS1, and Ki-67 in FAP patients. Original magnification: 200×. Immunostaining intensities are defined in Example 1.

FIG. 5. Involvement of platelets in colorectal cancer pathophysiology. (A) Platelet count was markedly elevated in FAP patients. Healthy subjects (n=16); FAP patients without colorectal cancer, aspirin nonusers (n=13); FAP patients without colorectal cancer, aspirin users (n=7); FAP patients with colorectal cancer, aspirin nonusers (n=12). Data are presented as means ±S.D. The asterisks (***) indicate a significant (p<0.001) increase compared with healthy control subjects. (B) Platelet count and circulating TXA₂ levels are positively correlated in FAP patients who are aspirin nonusers. Data were analyzed using Prism 5.0 statistical software (GraphPad Software, Inc., San Diego, Calif.).

FIG. 6. Circulating prostaglandin biosynthesis in healthy subjects, gastroesophageal reflux disease and Barrett's esophagus patients. 1=Healthy subjects (n=15); 2=gastroesophageal reflux disease patients (n=15); 3=Barrett's esophagus patients (n=15). Plasma samples were collected for measurement of circulating prostaglandin levels using an enzyme immunoassay kit (Cayman Chemical Co., Ann Arbor, Mich.). The asterisks indicate a significant (**, p<0.01; ***, p<0.001) difference compared to the group of healthy subjects.

### DETAILED DESCRIPTION OF ILLUSTRATIVE EMBODIMENTS

This disclosure describes methods of evaluating the risk that a subject may develop colorectal cancer (CRC). Colorectal cancer represents a common cause of cancer-related death in the United States. Lack of reliable biomarkers remains a challenge for early detection of colorectal cancer. Although colonoscopy screening and fecal occult blood testing have proven to be effective in the early detection of colorectal cancer, patient compliance is still low. Therefore, an urgent need exists to identify reliable biomarkers for early detection of colorectal cancer that can improve patient compliance.

This disclosure describes using circulating prostaglandin (PG) biosynthesis to assess colorectal cancer risk. Profiles of circulating prostaglandins (PGs) and platelet counts were determined in healthy (n=16), familial adenomatous polyposis (FAP) patients who were classified as regular aspirin users (n=14) or nonusers (n=24), and colorectal cancer patients with (n=18) or without FAP history (n=20). Immunohistochemistry staining was performed on biopsy samples. Profiles of circulating PG biosynthesis unexpectedly revealed that colorectal cancer progression is accompanied by a pronounced elevation of circulating thromboxane A2 (TXA₂) levels. A circulating TXA₂ level of 1000 pg/mL successfully identifies 95% of colorectal cancer patients. Further study suggested that the TXA₂ pathway is constitutively activated during colorectal tumorigenesis and is required for maintenance of the malignant characteristics of colon cancer cells. This disclosure therefore establishes the involvement of the TXA₂ pathway in colorectal cancer pathophysiology, and the utility of a TXA₂-targeting strategy for colorectal cancer early detection and management.

### Profiles of Circulating PG Biosynthesis in Colorectal Cancer

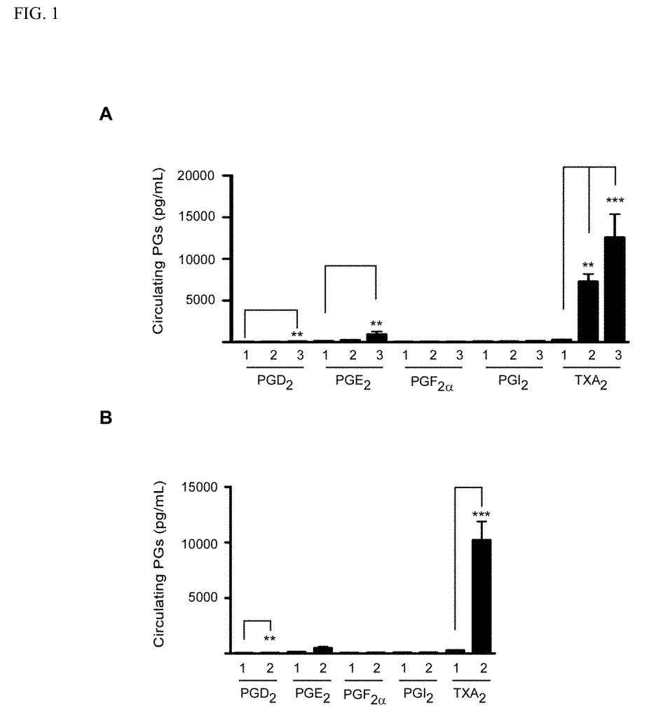

Circulating PG biosynthesis during colorectal cancer progression was profiled. The multistep nature of colorectal cancer (the so-called normal epithelial mucosa-adenoma-carcinoma sequence) has been well-established in FAP patients who universally develop colorectal cancer in the absence of colonic resection. Accordingly, FAP patients were recruited and further sub-grouped based on pathological disease stage. Among the five major bioactive PGs examined, TXA₂, but not PGE₂, was the most abundant PG in plasma from FAP patients (FIG. 1). Compared with healthy subjects, the levels of PGD₂, PGE₂, and TXA₂ were significantly elevated in FAP patients, whereas PGF_{2α}, and PGI₂levels did not change significantly. Circulating PGD₂ and PGE₂ were moderately elevated at the rather late stage (the adenoma-carcinoma sequence), whereas circulating TXA₂ was dramatically elevated throughout the entire progression of colorectal cancer in FAP patients. For example, in FAP patients who had developed colorectal cancer, circulating TXA₂ levels were increased to 44.3-fold of the normal level, but circulating PGE₂ levels were only increased by 6.7-fold.

Next, the profiles of circulating PG biosynthesis were analyzed in sporadic colorectal cancer patients. Similar results were obtained (FIG. 1B). Of the five PGs measured, TXA₂ was present at the highest concentration and only the levels of TXA₂ were significantly elevated in sporadic colorectal cancer patients compared with healthy subjects. The circulating TXA₂ levels in sporadic colorectal cancer patients were 35.9-fold higher than the normal level. These results indicate that, overall, colorectal cancer is accompanied by a pronounced elevation of the level of circulating TXA₂.

### Prognostic Value of Circulating TXA₂ Levels in Colorectal Cancer

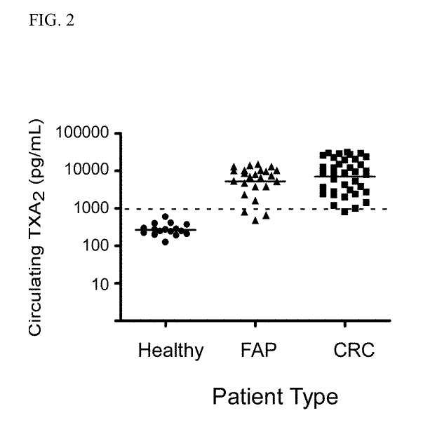

Circulating TXA₂ can predict the risk of developing colorectal cancer. To validate the prognostic value of circulating TXA₂ levels in colorectal cancer, a test study was conducted in both colorectal cancer patients and FAP patients. Results indicated that average circulating TXA₂ levels in healthy subjects were 284.2±112.0 pg/mL, whereas the average circulating TXA₂ levels in colorectal cancer patients and FAP patient were 11,328.3±9,701.3 and 7,275.4±4,438.6 pg/mL, respectively (FIG. 2). With a value of 1,000 pg/mL selected as a practical cutoff point to discriminate between colorectal cancer high-risk and low-risk groups, successful identification of colorectal cancer patients (36 of 38, 95%) and FAP patients (21 of 24, 88%) was achieved.

### Pathophysiological Role of the TXA₂ Pathway in Colorectal Cancer

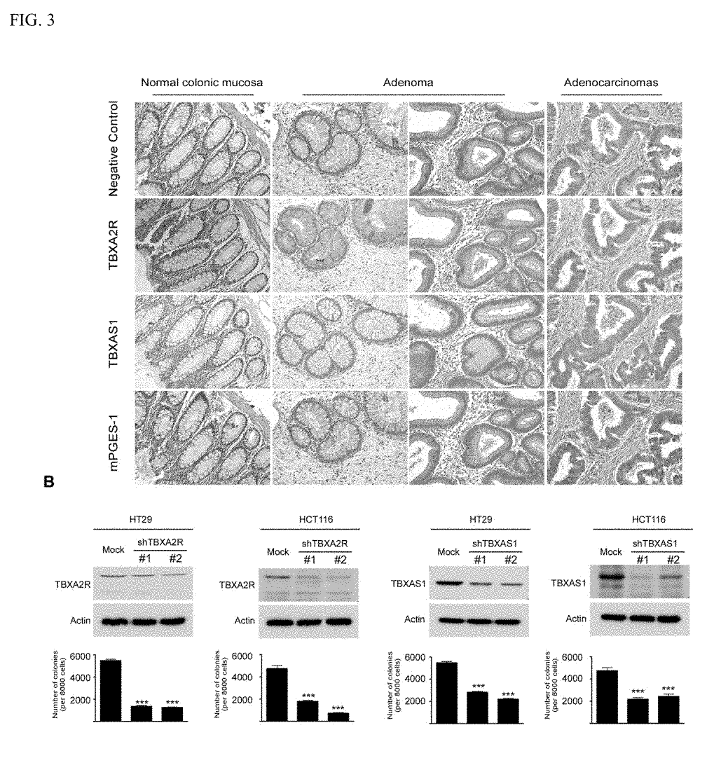

The TXA₂ pathway is involved in the development of colorectal cancer. The TXA₂ receptor (TBXA2R) and TXA₂ synthase 1 (TBXAS1, an enzyme involved in TXA₂ biosynthesis), were expressed biopsy samples (FIG. 3A). Immunohistochemistry staining showed that both TBXA2R and TBXAS1 were highly expressed in most colonic polyps or tumors, but not in normal colorectal tissues. Importantly, TBXA2R and TBXAS1 were co-localized with each other. Moreover, colorectal cancer progression can involve overexpression of microsomal prostaglandin E synthase-1 (mPGES-1, the rate-limiting enzyme for PGE₂ biosynthesis).

Next, whether the TXA₂ pathway is directly associated with tumorigenic properties of colon cancer cells was investigated. Anchorage-independent growth ability is an ex vivo indicator and a characteristic of the transformed cell phenotype. Knockdown of either TBXA2R or TBXAS1 in HT29 or HCT116 human colorectal cancer cells markedly inhibited their anchorage-independent cell growth ability (FIG. 3B).

### Aspirin Attenuates Colorectal Cancer in FAP Patients by Targeting the TXA₂ Pathway

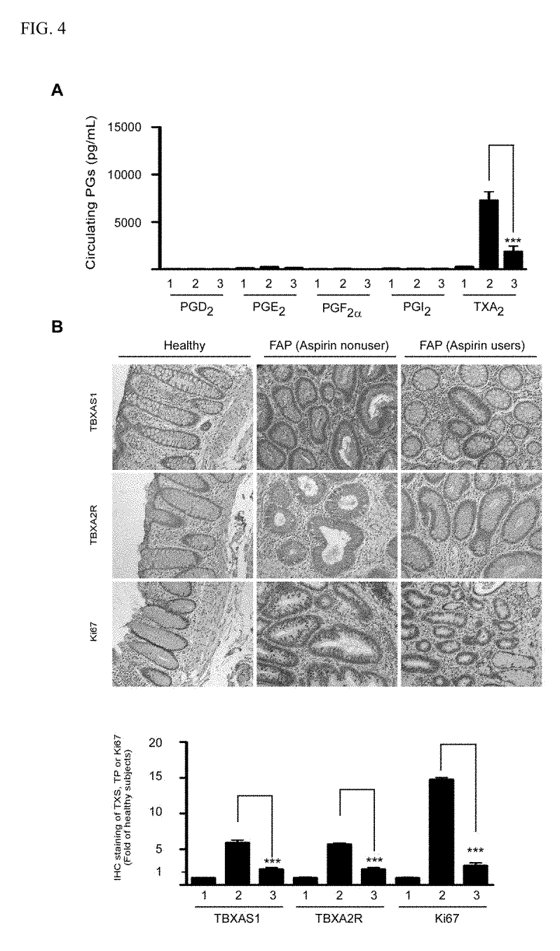

Aspirin can be a chemopreventive agent against colorectal cancer, but the molecular underpinnings of the activity of aspirin in the context of colorectal cancer progression remain imperfectly understood. Aspirin may reduce colorectal cancer risk by affecting the TXA₂ pathway. Regular use of aspirin was associated with significantly decreased circulating TXA₂ level in FAP patients, but had little effect on the levels of the other four PGs (FIG. 4A). Due to its very short half-life, TXA₂ primarily functions in an autocrine or paracrine manner by binding to TBXA2R, a typical G protein-coupled receptor (GPCR), which might signal platelet aggregation, cell growth, and/or migration. In this study, aspirin intake was associated with lower expression of TBXA2R, TBXAS1, and Ki-67 in epithelial cells from polyps (FIG. 4B).

Thus, this disclosure provides evidence that the TXA₂ pathway is constitutively activated during colorectal tumorigenesis and is involved in maintenance of the malignant characteristics of colon cancer cells. Importantly, colorectal cancer progression is associated with higher circulating TXA₂ levels, which may be a predictor of colon cancer risk.

Although a large body of evidence indicates that PGE₂ might be the predominant prostaglandin in cancer pathophysiology, the concept that PGE₂ is the only prostaglandin involved in carcinogenesis has long been challenged. For example, PGD₂ functions as a pro-resolution mediator in ulcerative colitis and PGI₂ is the major prostaglandin generated in ovarian epithelial cancer. This disclosure provides evidence that the TXA₂ pathway is involved in carcinogenesis and maintaining malignancy of colon cancer cells.

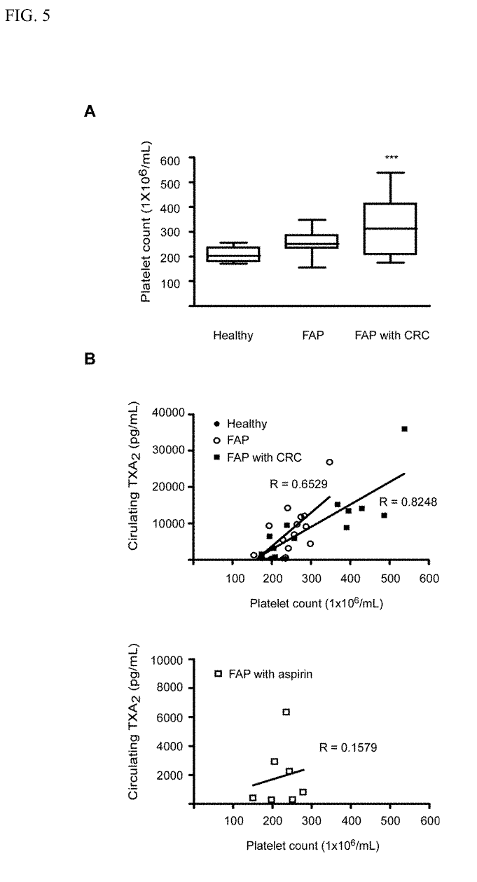

Platelets are a source of TXA₂ in blood. Thus, platelet count was examined and found to be markedly elevated in FAP patients, especially those who had already developed colorectal cancer (FIG. 5). Importantly, plasma TXA₂ levels positively correlated with platelet count in FAP patients who were aspirin nonusers, but was not associated with those patients who used aspirin regularly (FIG. 5). Overall, the results indicate that lowering circulating TXA₂ levels and/or interfering with the TXA₂ pathway may be a prophylactic and/or therapeutic strategy for colorectal cancer.

Thrombosis is a common complication in colorectal cancer patients, but its molecular mechanisms remain elusive. A dynamic balance between pro-thrombotic TXA₂ and anti-thrombotic PGI₂ production is generally accepted to be a contributor to homeostasis of the circulatory system. Elevated circulating TXA₂ levels, however, may be linked with colorectal cancer pathophysiology. In FAP patients, the levels of TXA₂ were increased by 25.6-fold compared to healthy subjects, whereas PGI₂levels did not change significantly. Thus, FAP patients might also be more prone to a risk of cardiovascular disease than healthy subjects.

Detecting malignant neoplasms at an early stage offers clinical advantages. However, very few reliable biomarkers are available to predict the risk of colorectal cancer, a common and deadly cancer. Considering the compliance issues associated with optical colonoscopy and the fecal occult blood test, the development of a reliable but minimally invasive method for colorectal cancer risk screening can improve compliance and, therefore, increase rates of early detection. Blood is easily sampled by relatively non-invasive methods and thus the introduction of a blood-based test could offer an advantage for enhancing patient compliance compared to other tests. Circulating TXA₂ levels can identify those at risk for colorectal cancer.

While described above in the context of an exemplary embodiment in which circulating TXA₂ is measured from blood drawn from subjects, circulating TXA₂ may be measured by any suitable method. For example, circulating TXA₂ may be measured by analyzing urinary TXA₂ metabolites such as 11-dehydro TXB₂, which might provide the best estimate of systemic TXA₂ biosynthesis in vivo.

Thus, this disclosure describes various methods that involve detecting an elevated level of TXA₂ in a biological sample obtained from a subject and/or measuring the level of circulating TXA₂ level in a biological sample obtained from a subject. The method may be used to detect, diagnose, and/or monitor the progression of colorectal cancer in the subject.

In one aspect, the method can include obtaining a biological sample from a subject having or at risk of having colorectal cancer, measuring circulating TXA₂ in the biological sample, and identifying the subject as having colorectal cancer if the circulating TXA₂ in the biological sample is greater than a predetermined level. As used herein, the term “at risk” refers to a subject that may or may not actually possess the described risk. Thus, for example, a subject “at risk” of having colorectal cancer is a subject possessing one or more risk factors associated with having colorectal cancer such as, for example, genetic predisposition, ancestry, age, sex, geographical location, lifestyle, or medical history, regardless of the subject manifests and symptoms or clinical signs of colorectal cancer. As used herein, “symptom” refers to any subjective evidence of disease or of a patient's condition, and “sign” or “clinical sign” refers to an objective physical finding relating to a particular condition capable of being found by one other than the patient.

Accordingly, the method may be performed using a biological sample from a subject before, during, or after the subject first exhibits a symptom or clinical sign of colorectal cancer. In cases where the method is performed after the subject first exhibits a symptom or clinical sign of colorectal cancer, the method may be used to monitor the progression of the disease and/or evaluate the efficacy of treatment by comparing a the circulating TXA₂ level in a present biological sample with the circulating TXA₂ level obtained from a previous biological sample. A medical professional can use the information regarding the progression or regression of the colorectal cancer to initiate, modify, change, terminate, or otherwise alter a course of treatment for the subject.

Treatment initiated before the subject first exhibits a symptom or clinical sign associated with colorectal cancer may result in decreasing the likelihood that the subject experiences clinical evidence of colorectal cancer compared to a similarly situated subject to whom treatment is not administered, decreasing the severity of symptoms and/or clinical signs of colorectal cancer experienced by the subject, and/or completely resolving the colorectal cancer. Treatment initiated after the subject first exhibits a symptom or clinical sign associated with colorectal cancer may result in decreasing the severity of symptoms and/or clinical signs of colorectal cancer compared to a similarly situated subject to whom treatment is not administered and/or completely resolving the colorectal cancer.

In some cases, the treatment can include administering to the subject a therapeutic that decreases circulating TXA₂.

Circulating TXA₂ may be measured directly by measuring the level of TXA₂ present in a sample that includes blood or a blood product such as, for example, plasma. Alternatively, circulating TXA₂ may be measured by measuring the level of a TXA₂ metabolite in a biological sample such as, for example, urine. An exemplary urinary TXA₂ metabolite is 11-dehydro TXB₂.

The predetermined level of circulating TXA₂ can be any level that provides a desired level of sensitivity and specificity in a given set of circumstances. Accordingly, the predetermined level of circulating TXA₂ can be a minimum of at least 700 pg/mL such as, for example, at least 750 pg/mL, at least 800 pg/mL, at least 850 pg/mL, at least 900 pg/mL, at least 950 pg/mL, or at least 1000 pg/mL. The predetermined level of circulating TXA₂ can be a maximum of no more than 1500 pg/mL, no more than 1400 pg/mL, no more than 1350 pg/mL, no more than 1300 pg/mL, no more than 1250 pg/mL, no more than 1200 pg/mL, no more than 1150 pg/mL, no more than 1100 pg/mL, no more than 1050 pg/mL, or no more than 1000 pg/mL. The predetermined level of circulating TXA₂ can be expressed as a range having endpoints defined by any minimum circulating TXA₂ level listed above and any maximum circulating TXA₂ level listed above that is greater than the minimum circulating TXA₂ level. In one particular exemplary embodiment, a predetermined level of circulating TXA₂ of 1000 pg/mL is sensitive enough to correctly identify 95% of subjects having colorectal cancer.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Materials and Methods

### Materials, Chemicals, and Reagents

Primary antibodies against human microsomal prostaglandin E synthase-1 (mPGES1), thromboxane A2 synthase 1 (TBXAS1), and thromboxane A2 receptor (TBXA2R) were obtained from Cayman Chemical Co. (Ann Arbor, Mich.). All chemicals were purchased from Sigma-Aldrich (St Louis, Mo.) unless otherwise specified.

### Cell Culture and Transfection

All cell lines used in this study were obtained from the American Type Culture Collection (ATCC, Manassas, Va.) and maintained following ATCC instructions. Cells were cytogenetically tested and authenticated before being frozen. Each vial of frozen cells was thawed and maintained for a maximum of 20 passages. For lentiviral transfection, the jetPEI reagent (Qbiogene, Inc., Montreal, Quebec, Canada) was used, following the manufacturer's instructions. The 29-mer small hairpin RNA (shRNA) constructs against human TBXA2R and TBXAS1 were obtained from Open Biosystems, Inc. (Huntsville, Ala.).

### Anchorage-Independent Cell Growth

In each well of a 6-well plate, cells (8×10³) were suspended in Basal Medium Eagle medium (1 mL, with 10% FBS and 0.33% agar) and plated over a layer of solidified BME medium (3 mL, with 10% FBS and 0.5% agar). The cultures were incubated in a 37° C., 5% CO₂ incubator for seven days and colonies in soft agar were counted under a microscope equipped with the Image-Pro Plus software program (Media Cybernetics, Bethesda, Md.).

### Western Blot Analysis

Protein samples (20 μg) were resolved by SDS-PAGE and transferred to Hybond C nitrocellulose membranes (Amersham Corporation, Arlington Heights, Ill.). After blocking, the membranes were probed with primary antibodies (1:1000) overnight at 4° C. The targeted protein bands were visualized using an enhanced chemiluminescence reagent (Amersham Corporation, Arlington Heights, Ill.) after hybridization with a secondary antibody conjugated with horseradish peroxidase.

### Patients

All clinical studies using human subjects or human materials were approved by the Mayo Clinic review board. Volunteers were recruited by the Gastroenterology and Hepatology group at Mayo Clinic, Rochester, Minn. Individuals in the healthy control group (n=16) were normal patients who underwent colonoscopy screening. Familial adenomatous polyposis (FAP) patients who reported taking two or more standard (325 mg) aspirin tablets per week were classified as regular aspirin users (n=14) and those reporting consumption of less aspirin were classified as aspirin nonusers (n=24) (Chan et al., 2007. N Engl J Med 356 (21):2131-2142). Individuals in the sporadic colorectal cancer group (n=20) were patients who were diagnosed with colorectal cancer, but without a family history of colorectal cancer. Other inclusion criteria were as follows: age at 18-75 years old; gender ratio approximately 1:1; and a non-smoking history.

### Measurement of Plasma Prostaglandin (PG) Levels

The measurement of PGs in plasma from patients was performed using enzyme immunoassay kits from Cayman Chemical Co. (Ann Arbor, Mich.) following the manufacturer's instructions. Briefly, blood was collected from a vein in the arm just inside the elbow using a 22 gauge needle. Before blood collection, the tourniquet was applied about three inches above the selected puncture site. Venous blood was drawn into a VACUTAINER plasma separation tube (#367964, BD Biosciences, Franklin Lakes, NJ) containing lithium heparin. Blood samples were then centrifuged at 2000×g for 15 minutes and the resulting supernatant fraction was designated as plasma to be used for prostaglandin measurement.

### Histology and Immunohistochemistry

Surgically resected human colon tissues at all clinical stages were fixed in 10% formalin overnight at room temperature. For histology, fixed tissues were embedded in paraffin, sectioned at 5 μm, and stained with haematoxylin and eosin (H&E) according to standard protocols. Immunohistochemistry staining for human mPGES1 (#160140, Cayman Chemical Co.; dilution 1:50), TBXAS1 (#160715, Cayman Chemical Co.; dilution 1:50), TBXA2R (#10004452, Cayman Chemical Co.; dilution 1:50), or Ki-67 (RM-9106, Thermo Scientific, Fremont, Calif.; dilution 1:200) was performed using an ABC complex kit (PK-6100, Vector Laboratories, Burlingame, Calif.) following the manufacturer's instructions. Sections were counterstained with Harris's haematoxylin. For antibody-negative controls, the primary antibodies were substituted with normal rabbit serum. Immunohistochemistry staining intensity was quantified by calculating the integrated optical density (IOD, sum) of area of interest using the Image Pro-Plus 7.0 software program (Media Cybernetics, Inc., Rockville, Md.).

### Statistical Analysis

Statistical analysis was performed using the Prism 5.0 statistical software package (GraphPad Software, Inc., San Diego, Calif.). The Turkey's t-test was used to compare data between two groups. One-way ANOVA and the Bonferroni correction were used to compare data between three or more groups. Values are expressed as means ±S.D. and a p value of <0.05 was considered statistically significant.

### Example 2

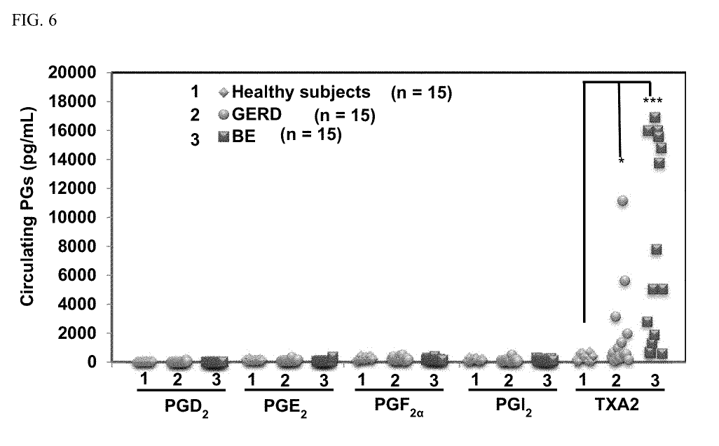

Briefly, blood was collected from a vein in the arm just inside the elbow using a 22 gauge needle. Before blood collection, the tourniquet was applied about three inches above the selected puncture site. Venous blood was drawn into a VACUTAINER plasma separation tube (BD Biosciences, Franklin Lakes, N.J.) containing lithium heparin. Blood samples were then centrifuged at 2000×g for 15 minutes and the resulting supernatant fraction was designated as plasma. Plasma prostaglandins were measured using enzyme immunoassay kits from Cayman Chemical Co. (Ann Arbor, Mich.) following the manufacturer's instructions. Since PGD₂, PGF_{2α}, PGI₂, and TXA₂ are unstable in vivo, the circulating level of each was determined by measuring a plasma metabolite: 11-beta-PGF_{2α}, (PGD₂), 13,14-dihydro-15-keto-PGF_{2α}, (PGF_{2α}), 6-keto-PGF_{1α}, (PGI₂), and TXB₂ (TXA₂). Results are shown in FIG. 6.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method comprising:
obtaining a biological sample from a subject having or at risk of having colorectal cancer; and
measuring circulating TXA₂ in the biological sample.

2. The method of claim 1 wherein measuring circulating TXA₂ in the biological sample comprises determining the amount of TXA₂ in a biological sample comprising plasma.

3. The method of claim 1 wherein measuring circulating TXA₂ in the biological sample comprises determining the amount of a TXA₂ metabolite in a biological sample comprising urine.

4. The method of claim 3 wherein the TXA₂ metabolite comprises 11-dehydro TXB₂.

5. The method of claim 1 wherein measuring circulating TXA₂ in the biological sample comprises determining whether circulating TXA₂ is at least 1000 pg/mL.

6. The method of claim 1 further comprising administering therapy to the subject effective for treating colorectal cancer.

7. The method of claim 6 wherein the therapy is effective to decrease circulating TXA₂.

8. A method comprising:
obtaining a present biological sample from a subject having colorectal cancer;
measuring present circulating TXA₂ in the biological sample;
obtaining a previous circulating TXA₂ value obtained from a previous biological sample obtained from the subject; and
detecting a change in circulating TXA₂ between the previous biological sample and the present biological sample.

9. The method of claim 8 wherein the subject has undergone therapeutic treatment for colorectal cancer between obtaining the previous biological sample and obtaining the present biological sample.

10. The method of claim 7 further comprising administering therapy to the subject effective for treating colorectal cancer.

11. The method of claim 8 wherein the therapy is effective to decrease circulating TXA₂.

12. The method of claim 8 wherein measuring circulating TXA₂ in the present biological sample comprises determining the amount of TXA₂ in a present biological sample comprising plasma.

13. The method of claim 8 wherein measuring circulating TXA₂ in the present biological sample comprises determining the amount of a TXA₂ metabolite in a present biological sample comprising urine.

14. The method of claim 13 wherein the TXA₂ metabolite comprises 11-dehydro TXB₂.
